(19) United States
(12) Patent Application Publication
(10) Pub. No.: US 2005/0049504 A1
(43) Pub. Date: Mar. 3, 2005

(54) ULTRASONIC VEIN DETECTOR AND RELATING METHOD

(76) Inventors: Meng-Tsung Lo, Taipei Hsien (TW); Chung-Yuo Wu, Taipei Hsien (TW); Yi-Chung Chang, Taipei Hsien (TW)

Correspondence Address: (NAIPC) NORTH AMERICA INTERNATIONAL PATENT OFFICE, P.O. BOX 506, MERRIFIELD, VA, 22116, US

(21) Appl. No.: 10/708,398

(22) Filed: Mar. 1, 2004

(30) Foreign Application Priority Data
Aug. 27, 2003 (TW) 092123596

(51) Int. Cl. A61B 8/02

(52) U.S. Cl. 600/453

(57) ABSTRACT

An ultrasonic vein detector for detecting the position of a vein in a specific part of an examinee includes an ultrasonic emitter having an oscillator for generating indicative pulse ultrasonic signals toward the examinee, a pulse presser for applying pulse stress signals in a frequency different from the heartbeat of the examinee, an ultrasonic sensor for sensing reflected waves of the indicative pulse ultrasonic signals on every reflecting point and converting them into electrical signals, and a microprocessor for receiving the electrical signals from the ultrasonic sensor and calculating the Doppler shift of the electrical signals generated from the reflected waves in order to find the reflecting points corresponding to the pulse stress signals.

### BACKGROUND OF INVENTION

1. Field of the Invention

The invention relates to an ultrasonic vein detector and its related method, and more particularly to an ultrasonic vein detector and its related method utilizing ultrasonic signals to detect the position of a vein.

2. Description of the Prior Art

Generally performed medical services such as endoscope operations or radiation tumor treatments are accompanied with objective data-gathering means such as X-ray scan or magnetic resonance imaging (MRI) in order to provide objective images to the operator in order to aid the operation.

On the other hand, blood tests and injections only depend on the experience of examiners to select a proper position to insert the needle to extract a blood sample or inject medicine; no objective references are used. In the case of the examinees being the fat or the infant, the task is made more difficult because their vein is not always easily found, meaning that the examiner may have to repeatedly insert the needle until the proper position for injection is found. That not only causes pain to the examinees but also raises the risk. Therefore, an objective reference of the position of veins is necessary to increase the quality of medical treatments.

However, the compositions of blood, a blood vessel, and the surrounding soft tissues such as muscles and organs are similar so that they can hardly be distinguished by X-ray. MRI is one solution, but since it requires an intense magnetic field (up to 1 Tesla), a heavy electromagnet, and a large cooler, it is difficult for the MRI to come into wide use. Therefore blood current, detected by emitting ultrasonic wave into the examinee 8 with an ultrasound probe 9 and measuring its echo as shown in FIG. 1, is used to determine the position of the vein.

In fact, the reflection values of blood and soft tissues do not differ so much from each other. Therefore, it is difficult to generate a reflective signal at the interface between blood and soft tissues. FIG. 1 shows a conventional ultrasound probe for detecting blood current. The conventional ultrasound probe makes use of the Doppler effect. When a blood cell 80 moves toward an ultrasonic emitter 91 as in FIG. 2, the reflected signal sensed by a sensor 92 shifts to a higher frequency, and when the blood cell 80 moves away from the ultrasonic emitter 91 as in FIG. 3, the sensor 92 senses a lower frequency. Such phenomenons are well known as Doppler effect. Therefore, a Doppler shift blood current diagram in FIG. 4 can be measured by the method according to the prior art. According to the horizontal time axis in the diagram, the obvious relationship between the blood current diagram and an electrocardiogram above it is shown.

However, with the insertion of a needle, it is required to select a vein with low current speed for blood test or injection, meaning that the Doppler shift effect is generally unobvious; added, it is difficult to distinguish the blood from neighboring soft tissues. Furthermore, continuous ultrasonic detecting can only tell the operator whether there is a moving object but cannot provide any axial analysis, i.e. the depth of the moving object remains unknown. Therefore, it is still an object to improve the conventional technology for a better medial care.

### SUMMARY OF INVENTION

It is therefore a primary objective of the claimed invention to provide an ultrasonic vein detector capable of detecting blood. The device according to the present invention is low-cost and simply-structured. The present invention also provides a method for correctly detecting a vein by the ultrasonic vein detector.

Briefly, an ultrasonic vein detector for detecting the position of a vein in a specific part of an examinee includes an ultrasonic emitter having an oscillator for generating indicative pulse ultrasonic signals toward the examinee, a pulse presser for applying pulse stress signals in a frequency different from the heartbeat of the examinee, an ultrasonic sensor for sensing reflected waves of the indicative pulse ultrasonic signals on every reflecting points and converting them into electrical signals, and a microprocessor for receiving the electrical signals from the ultrasonic sensor and calculating the Doppler shift of the electrical signals generated from the reflected waves in order to find the reflecting points corresponding to the pulse stress signals.

The present invention also provides a method for detecting the position of a vein in a specific part of an examinee by ultrasonic waves including (a) emitting an indicative pulse ultrasonic signal toward the examinee from an emitting point, (b) applying pulse stress signals in a frequency different from the pulse ultrasonic signal and the heartbeat of the examinee, on the examinee, (c) sensing a reflected wave of the indicative pulse ultrasonic signal and converting it into an electrical signal, and (d) calculating the Doppler shift of the electrical signal generated from the reflected wave in order to find the reflecting point corresponding to the pulse stress signal.

These and other objectives of the present invention will no doubt become obvious to those of ordinary skill in the art after reading the following detailed description of the preferred embodiment that is illustrated in the various figures and drawings.

### BRIEF DESCRIPTION OF DRAWINGS

FIG. 1 illustrates a conventional ultrasound probe detecting blood current.

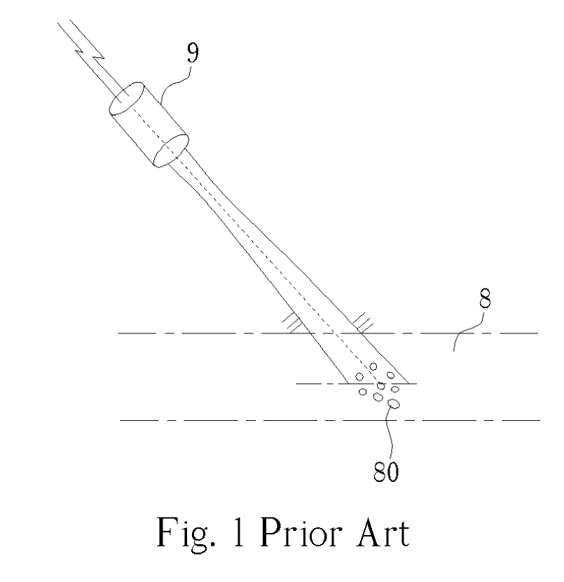

FIG. 2 illustrates an ascending shift of the frequency of the reflective ultrasonic signals according to the Doppler effect when the object moves toward the ultrasound probe.

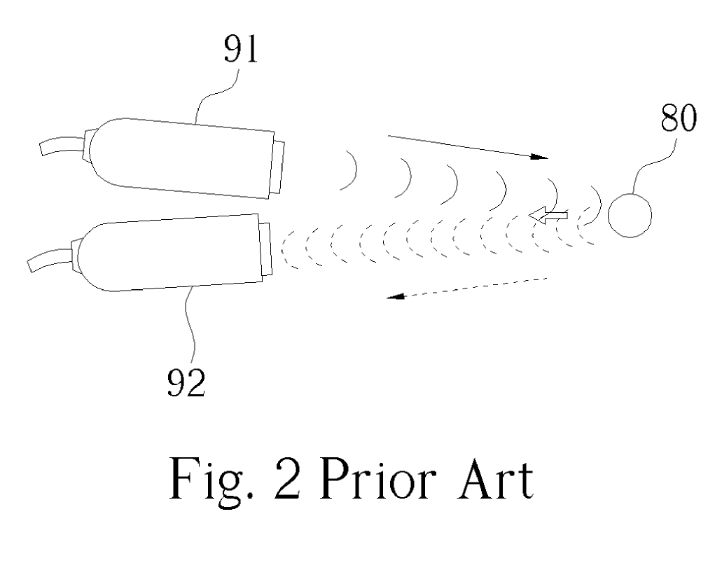

FIG. 3 illustrates a descending shift of the frequency of the reflective ultrasonic signals according to the Doppler effect when the object moves away from the ultrasound probe.

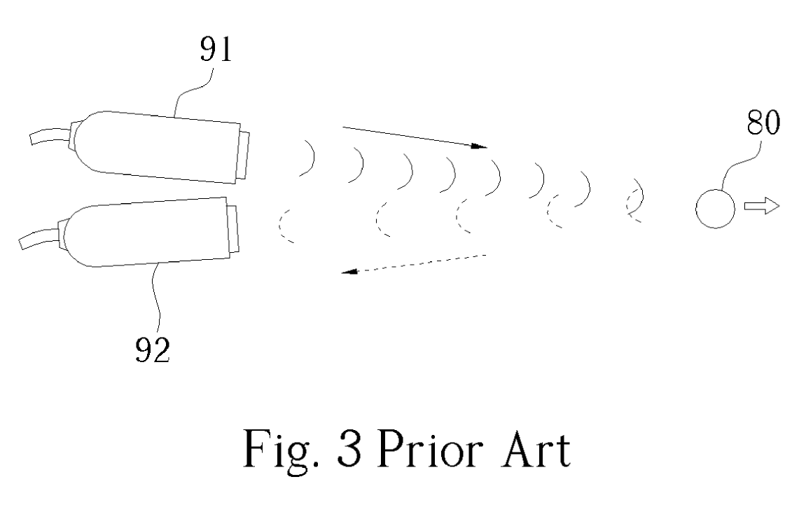

FIG. 4 is a timing diagram comparing the Doppler shift of the blood current measured by the ultrasonic signals with an electrocardiogram.

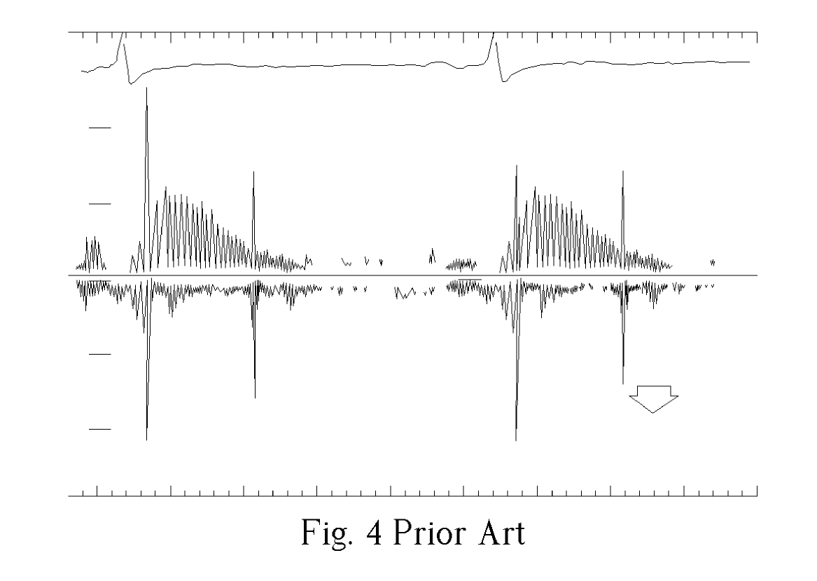

FIG. 5 is a block diagram of an ultrasonic vein detector according to the present invention.

FIG. 6 illustrates the ultrasonic vein detector applied on the arm of the examinee.

FIG. 7 is a flowchart of the method for detecting the vein.

FIG. 8 is a timing diagram illustrating the relationship between the Doppler shift of the arterial and venous blood current measured by the ultrasonic vein detector and the stress signals.

FIG. 9 illustrates the method for detecting the vein by calculating the echoes.

### DETAILED DESCRIPTION

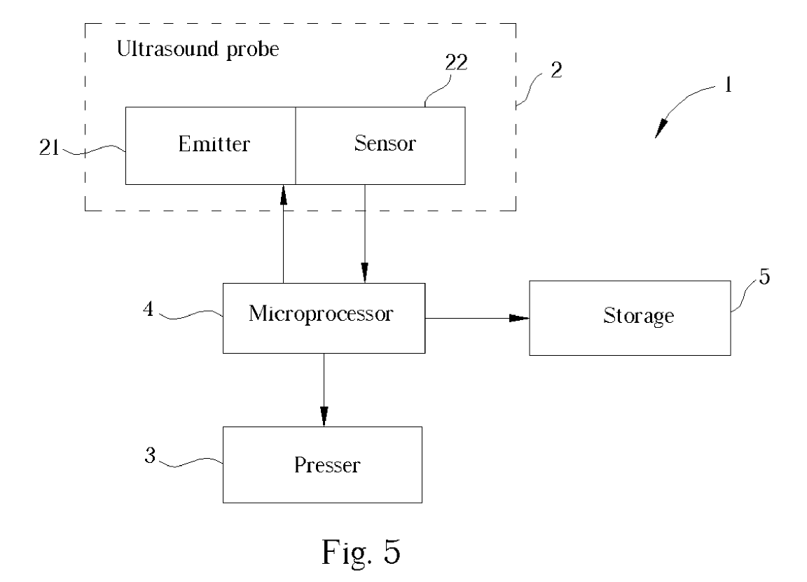

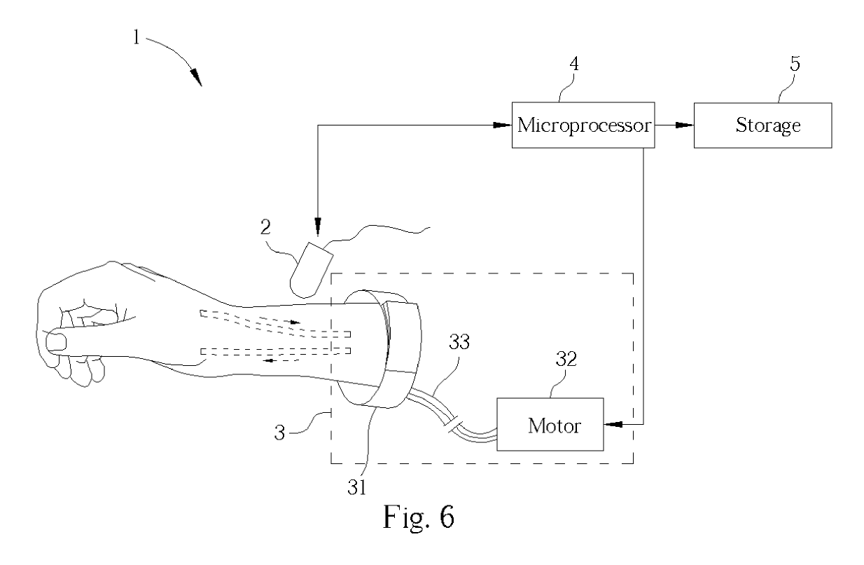

Please refer to FIG. 5 and FIG. 6; an ultrasonic vein detector 1 according to the present invention includes a pulse ultrasonic emitter 21, an ultrasonic sensor 22, a pulse presser 3, a microprocessor 4, and a storage 5.

An ultrasound probe 2 includes the pulse ultrasonic emitter 21 and the ultrasonic sensor 22. The emitter 21 has an oscillator made of piezoelectric materials so that the emitter 21 generates discontinuous indicative pulse ultrasonic signals when receiving driving signals from the microprocessor 4.

The pulse presser 3 in this embodiment includes a blood pressure belt 31 and a motor 32. The belt 31 is composed of an airbag (not shown) connected to the motor 32 by a tube 33. The motor 32 inflates and deflates the airbag by the tube 33 according to the command by the microprocessor 4.

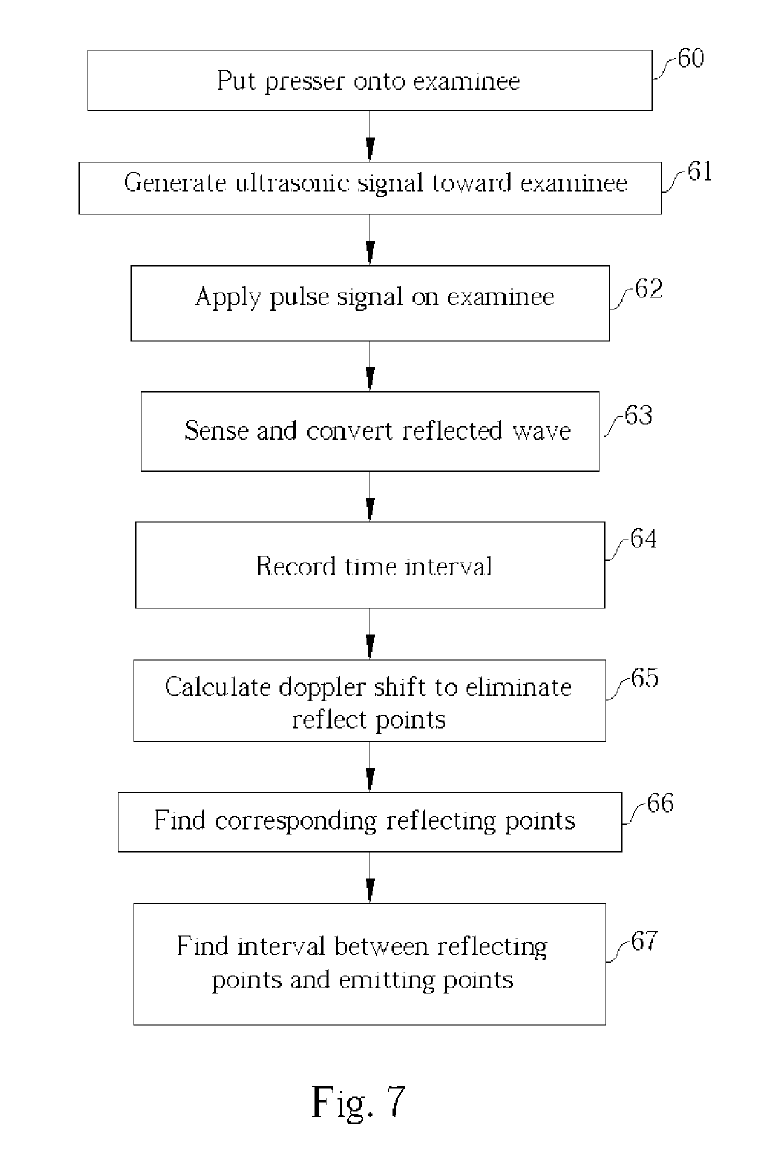

As shown in FIG. 7, in order to find a vein in a particular part of an examinee such as his arm, first tie the belt 31 of the presser 3 around the examinees arm as in Step 60. Then the microprocessor 4 commands the emitter 21 to generate discontinuous pulse ultrasonic signals at a certain frequency e.g. 10 MHz toward the arm as in Step 61. At the same time, the microprocessor 4 commands the motor 32 to inflate the airbag so that the belt 31 applies pulse stress signals to the arm.

Bearing in mind that the pressure pressing blood back to the heart in a vein is much less than that in an artery, it is recommended that the pressure of the belt 31 be kept between the venous pressure and the diastolic pressure of the examinee for the purpose of stopping the current in the vein while keeping the current in the artery in order to distinguish the artery from the vein. Additionally, in order to distinguish the stress signals from heartbeat, the stress signals are applied in a different frequency from that of heartbeat.

The ultrasonic sensor 2 within the ultrasound probe 2 then senses the echoes of the ultrasonic pulse signals and converts them into electrical signals as in Step 63. Of course, the echoes include those reflected from the interfaces between the air and skin, blood and blood vessel, and muscles and bones.

The storage 5 first records the time when the indicative ultrasonic signal is emitted and then records the time intervals between reflected waves as also referred to as received reflecting signals as in Step 64.

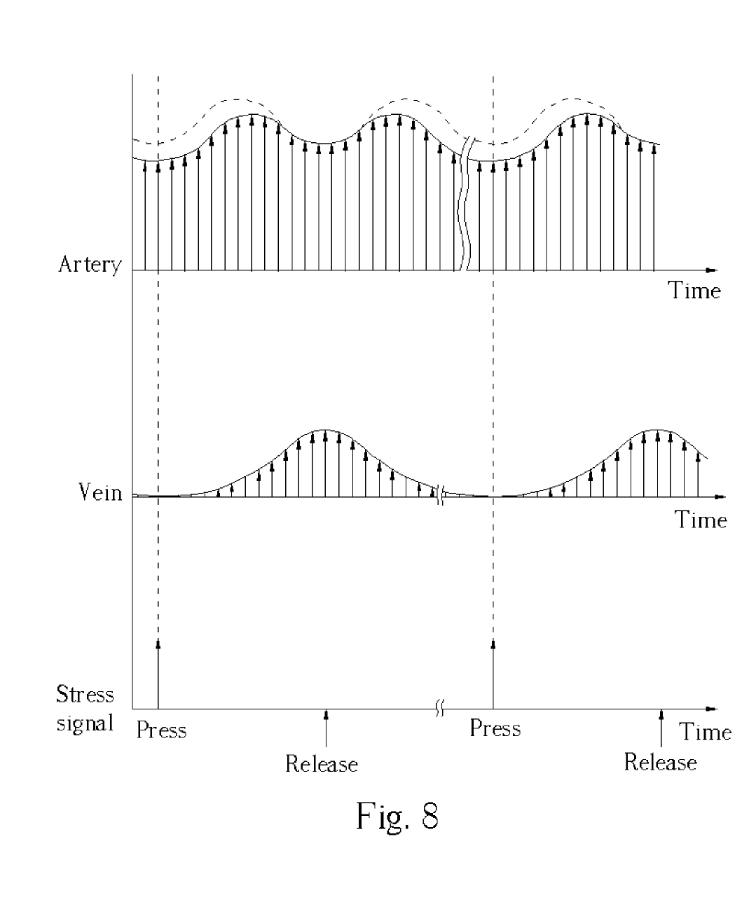

In Step 65, the microprocessor 4 calculates the Doppler shift of the reflected waves. Echoes reflected from tissues such as the bones, skin, and muscles are not time-variety direct current (dc) signals, meaning that there is no Doppler shift. As shown in FIG. 8, the Doppler shift of arterial blood relates to the heartbeat, and the blood current is not stopped by the stress signals; thus the effective reflection of arterial blood experiences under the Doppler shift is stronger and more continuous than that of venous blood. On the other hand, the effective reflection of venous blood experiences under the Doppler shift is closely related to the pulse stress signals. Therefore, those reflecting points that is not corresponding to the pulse stress signals can be eliminated by detecting whether the pulse stress signals exit. In step66, the reflecting points that is corresponding to the pulse stress signals can be found out, i.e. the reflection of the blood in the vein.

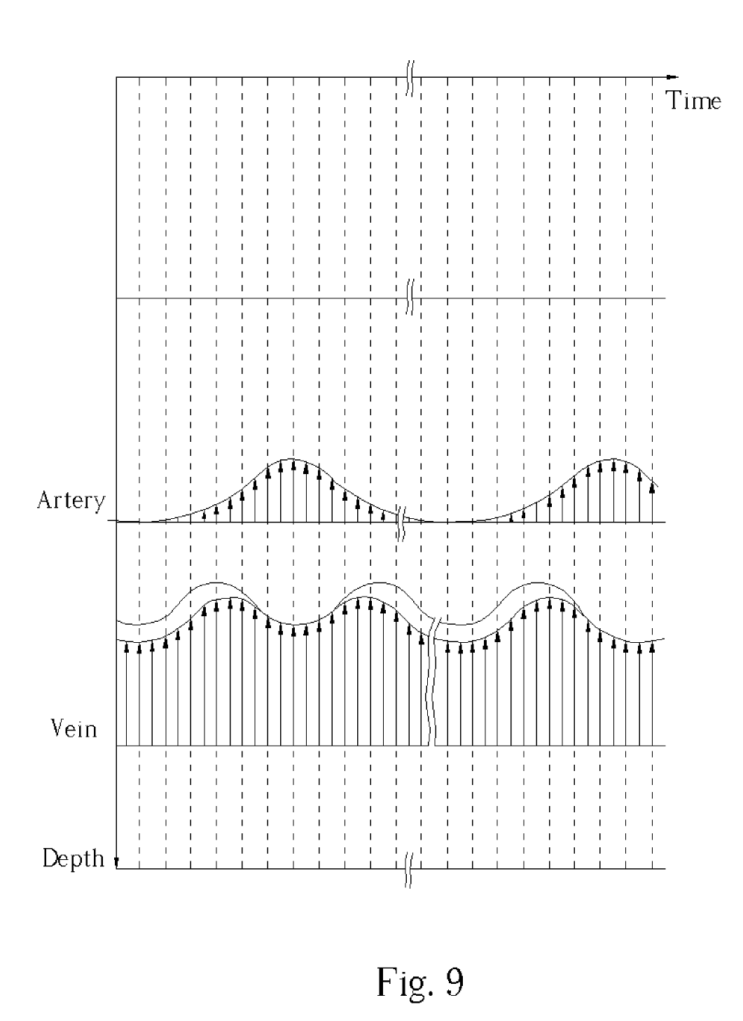

Finally in Step 67, accumulate the echoes in a period of time as in FIG. 9. As described above, the vein and the artery have different echo modes, so they can be distinguished by the modes and types of the echoes. The microprocessor 4 subtracts the time of signal emission from the time of signal reflection for time intervals between signals and their echoes, and then multiplies the time intervals by the ultrasonic transmission speed to obtain the interval distance between the reflecting points and emitting points. Since the wave transmission speed in the air medium is about 330 m/sec, in a condition that the analyzable frequency of electrical signals is in Giga Hz order, the spatial resolution can reach micron level.

In contrast to the prior art, the ultrasonic vein detector according to the present invention utilizes a presser to lower the blood current in a vein to cause the Doppler shift of the incident ultrasonic echoes in order to distinguish the vein from an artery, other soft tissues and bones. Specifically, the present invention utilizes the pulse ultrasonic emitter to detect along a specific direction so that the echoes can be regarded as recorded data along a time axis, and the position of the vein can be found by accumulating those data. The present invention can be applied even if the examinee is the fat or the infant.

Those skilled in the art will readily observe that numerous modifications and alterations of the device may be made while retaining the teachings of the invention. Accordingly, the above disclosure should be construed as limited only by the metes and bounds of the appended claims. Especially, those skilled in the art know that the steps from Step 65 to Step 67 proceed almost instantly.

1. An ultrasonic vein detector for detecting the position of a vein in a specific part of an examinee comprising:
an ultrasonic emitter having an oscillator for generating indicative pulse ultrasonic signals toward the examinee;
a pulse presser for applying pulse stress signals to the part of the examine, wherein the frequency of the signal is different from the heartbeat frequency of the examinee;
an ultrasonic sensor for sensing the back waves which is the reflection of the indicative pulse ultrasonic signals hitting every reflecting point of the part of the examinee, and converting them into electrical signals; and
a microprocessor for receiving the electrical signals from the ultrasonic sensor and calculating the Doppler shift of the electrical signals generated from the back waves in order to find the reflecting points corresponding to the pulse stress signals.

2. The ultrasonic vein detector of claim 1 further comprising a storage for storing the electrical signals outputted by the ultrasonic sensor.

3. A method for detecting the position of a vein in a specific part of an examinee by ultrasonic waves comprising:
(a) emitting an indicative pulse ultrasonic signal toward the examinee from an emitting point;
(b) applying pulse stress signals on the examinee, wherein the frequency of the pulse stress signals is different to the frequency of the pulse ultrasonic signal and the heartbeat of the examinee;
(c) sensing a back wave which is the reflection of the indicative pulse ultrasonic signals hitting from the part of the examinee and converting it into an electrical signal; and
(d) calculating the Doppler shift of the electrical signal generated from the back wave in order to find the reflecting point corresponding to the pulse stress signal.

4. The method of claim 3 wherein the pulse stress signal is non-periodical.

5. The method of claim 4 wherein in step (d) a time interval between the emitting the indicative pulse ultrasonic signal and sensing the back wave is recorded and the time interval is multiplied by the ultrasonic transmission speed in order to obtain the interval distance between a reflecting point and an emitting point.
